(19) United States
(12) Patent Application Publication
(10) Pub. No.: US 2007/0173726 A1
(43) Pub. Date: Jul. 26, 2007

(54) Bio-signal detection device and method of managing health of user using the device

(75) Inventors: Kyung Ho Kim, Yongin-si (KR); Hong Sig Kim, Seongnam-si (KR); Sang Kon Bae, Seoul (KR); Kun Kook Park, Suwon-si (KR); Kwang Hyeon Lee, Yongin-si (KR); Woo Young Jang, Seoul (KR); Jeong Je Park, Daegu (KR); Hye Jin Jung, Seoul (KR); Jae Chan Park, Yongin-si (KR); Jin Sang Hwang, Suwon-si (KR); Soo Kwan Kim, Seongnam-si (KR); Eun Young Choe, Suwon-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/492,072

(22) Filed: Jul. 25, 2006

(30) Foreign Application Priority Data
Sep. 9, 2005 (KR) 10-2005-0084320

(51) Int. Cl. A61B 5/02; A61B 5/00; A61B 5/05

(52) U.S. Cl. 600/483; 600/347; 600/485; 600/490; 600/301; 128/920; 128/921

(57) ABSTRACT

A bio-signal detection device and a method of managing the health of a user is used with a health management system. The device includes a main body having palm support portions formed on the top of both sides thereof, a sensing unit formed on at least one of the palm support portions to detect a bio-signal of a user and including electrodes, and an operation unit receiving a signal from the sensing unit to operate. The method includes maintaining a first and second database including inquiry information and information on the normal physical condition of a user; comparing a measured present physical condition with the normal physical condition and detecting a changed item associated with the physical condition of the user; outputting lifestyle inquiry information associated with the detected changed item; receiving a reply to the outputted lifestyle inquiry information; analyzing a health condition of the user; and reporting health condition information.

### CROSS-REFERENCE TO RELATED APPLICATIONS

This application claims the benefit of Korean Patent Application No. 2005-84320, filed on Sep. 9, 2005, in the Korean Intellectual Property Office, the disclosure of which is incorporated herein in its entirety by reference.

### BACKGROUND OF THE INVENTION

1. Field of the Invention

The present invention relates to a method and system for managing health, and more particularly, to a method and system for managing health by using lifestyle inquiry information according to a change in the physical condition of a user.

2. Description of the Related Art

As society has become more complicated and the level of lifestyles becomes higher, concerns about health have increased. Adult diseases have increased due to changes in diet, lack of exercise, and increase of stress. Accordingly, a bio-signal measurement device that can measure not only body weight but also various bio-signals including measuring electrical activity of the heart through an electrocardiogram has been developed and sold at market.

An example of the device is shown in FIGS. 1 and 2. FIG. 1 is a front view of a bio-signal measurement device 10 disclosed in Korean Patent No. 159588, and FIG. 2 is a perspective view illustrating a state of using the device. As illustrated, grip portions 12 and 13 installed at a housing 11 and to be gripped by hands, a display unit 16 installed on the surface of the housing 11 and displaying health management information, and other operation switches 15a, 15b, 15c, and 15d are included. Also, in the housing 11, a circuit formed of an electronic element such as an operation means computing information required in managing health based on the impedance of a body or body-specific information is included.

The grip portions 12 and 13 extending right and left have a cylindrical shape, electrodes 17 and 18 formed in the shape of a ring and applying a constant current are disposed on the grip portions 12 and 13, electrodes 19 and 20 formed in the shape of a ring and detecting voltage are separated by separation portions 12b and 13b, and grip portions 12a, 12c, 13a, and 13c for determining a position are installed at both ends of both grip portions 12 and 13.

As shown in FIG. 2, the user grips the horizontal grip portions 12 and 13 while the user stands and extends the arms at shoulder width, and the bio-signal measurement device 10 formed as described above measures a bio-signal through each of the electrodes 17, 18, 19, and 20. Since a measured value changes according to the posture of the user, i.e., if the user folds his arm, and a measurement position, it is important that the posture of the user is precisely maintained and a position of the electrodes attached to the measurement device 10 is precisely maintained. Also, the bio-signal measurement device measures bio-signals such as body fat and basal metabolic rate, by reading the signals through both hands.

The problems of the bio-signal measurement device as described above are as follows.

Since the user stands and grips the electrodes while using the device, it is difficult to maintain a state in which the user stands with extended arms for a long time. Also, since the user grips the electrodes while using the device, a measurement value may change according to the amount of force used in the grip. Accordingly, a bio-signal cannot be accurately measured.

As bio-signals capable of being measured are limited, precise diagnostic information cannot be provided. As an example, a blood pressure cuff for measuring blood pressure is not included, so therefore it is impossible to measure blood pressure.

Also, since there is no unit included that is able to fix a grip to the electrodes, the position in which the electrodes come into contact with a palm changes during every use and therefore a bio-signal can-not be accurately measured.

Further, there is a problem with an inquiry method for managing health using the bio-signal measurement device 10. As a representative example of a conventional health management apparatus, the apparatus indicates only the degree of obesity, an ideal body weight and the recommended one-day calorie intake for the ideal body weight, the number of calories of food ingested for one day, and the number of calories burned for one day, of a user. However, the conventional health management apparatus subtracts the number of calories burned by the user from the number of calories of food ingested by the user, changes the result of the subtraction into body weight, and simply notifies the user of that body weight. Therefore, detailed information on how to achieve an ideal body weight of the user is not provided.

A conventional health management system provides an inquiry list associated with the user's health condition to the user and simple feedback information corresponding to the health condition of the user is matched according to a reply to the inquiry list.

As described above, the conventional health management system has a problem in that data corresponding to the user's present physical condition does not exist and the health condition of a user is determined by a simple inquiry, thereby not precisely checking the health condition of the user.

Also, since the conventional health management system includes duplicated inquiry information in an inquiry list for managing the health of the user, the user has to repeatedly reply to the duplicated inquiry information.

### SUMMARY OF THE INVENTION

To solve the problems of a conventional method as described above, one embodiment of the present invention provides a bio-signal measurement device that may accurately measure a bio-signal by fixing a position in which a palm is in contact with electrodes while the hands are open, and a health management method using the device.

In one aspect, the present invention also provides a bio-signal measurement device that provides electrodes capable of being kept in reliable contact with a palm and keeps a palm in reliable contact with the electrodes, and a health management method using the device.

One embodiment of the present invention also provides a bio-signal measurement device including a blood pressure cuff and that measures various bio-signals, including blood pressure, and a health management method using the device.

One aspect of the present invention also provides a bio-signal measurement device that can fix a position of a hand to reduce error due to a measurement position and a health management method using the device.

The present invention, in one aspect, also provides a bio-signal measurement device that determines the physical condition of a user via inquiries about the physical condition of the user and measures a bio-signal of the user, and a health management method using the device.

An embodiment of the present invention also provides a bio-signal measurement device that recognizes a change of the physical condition of a user and more precisely analyzes the health condition of the user according to an inquiry reply corresponding to the change, and a health management method using the device.

According to one aspect, the present invention also provides a bio-signal measurement device showing a more detailed health management scheme according to the analyzed health condition of a user and a health management method using the device.

The present invention, according to an embodiment, also provides a method and a system that clearly divides the diet, exercise, and activity scheme for managing the health of a user and shows feedback information associated with the lifestyle of the user.

In one aspect, the present invention also provides a method and system that determines the health condition of a user and displays the user's health condition information and a management scheme according to the user's health condition information.

According to an aspect of the present invention, there is provided a bio-signal detection device, including: a main body having palm support portions formed on both sides of the top thereof; a sensing unit formed on at least one of the palm support portions in order to detect a bio-signal of a user and including electrodes to become electrically in contact with a palm of the user; and an operation unit formed inside the main body and receiving a signal from the sensing unit to operate.

According to another aspect of the present invention, there is provided a bio-signal detection device detecting at least two bio-signals at the same time, including: a horizontal case horizontally formed; a vertical case vertically protruded from the center of the horizontal case; at least two electrodes formed on both sides of the horizontal case, respectively, to be able to come into contact with a palm; and a display unit installed in the vertical case.

According to still another aspect of the present invention, there is provided a bio-signal detection device, including: a main body; a palm support portion formed on the right and left of the main body; at least two electrodes formed on the top surface of the palm support portions and able to electrically come into contact with an palm of a user; a display unit installed in the main body; a blood pressure cuff installed on the palm support portion to be able to come into contact with a finger to measure blood pressure; and a finger guide formed on one of the palm support portions, opposite to the blood pressure cuff, and situated to be in between fingers to block forward movement of a finger.

According to yet another aspect of the present invention, there is provided a method of managing the health of a user in a health management system, including: maintaining a first database including inquiry information and a second database including information on a normal physical condition of a user; measuring the present physical condition of the user; comparing the measured present physical condition with the normal physical condition information by referring to the second database and detecting a changed item associated with the physical condition of the user; outputting lifestyle inquiry information associated with the detected changed item of stored inquiry information by referring to the first database; receiving information on a reply to the inquiry associated with the outputted lifestyle inquiry information from the user; analyzing a health condition of the user based on the measured present physical condition and the inputted inquiry reply information; and reporting the health condition information of the user according to the result of the analysis.

According to a further aspect of the present invention, there is provided a health management system including: a first database including and storing inquiry information; a second database including and maintaining normal physical condition information of a user; a measurement unit measuring the present physical condition of the user; a detection unit comparing the measured present physical condition with the normal physical condition information by referring to the second database and detecting a changed item associated with the physical condition of the user; an output unit outputting lifestyle inquiry information associated with the detected changed item from the inquiry information stored by referring to the first database; an input unit receiving inquiry reply information, in reply to the outputted lifestyle inquiry information, from the user; an analysis unit analyzing the health condition of the user based on the measured present physical condition and the inputted inquiry reply information; and a report unit reporting health condition information of the user according to a result of the analysis.

Additional aspects and/or advantages of the invention will be set forth in part in the description which follows and, in part, will be apparent from the description, or may be learned by practice of the invention

### BRIEF DESCRIPTION OF THE DRAWINGS

The above and other features and advantages of the present invention will become more apparent by describing in detail exemplary embodiments thereof with reference to the attached drawings in which:

FIG. 1 is a front view of a bio-signal measurement device of the prior art;

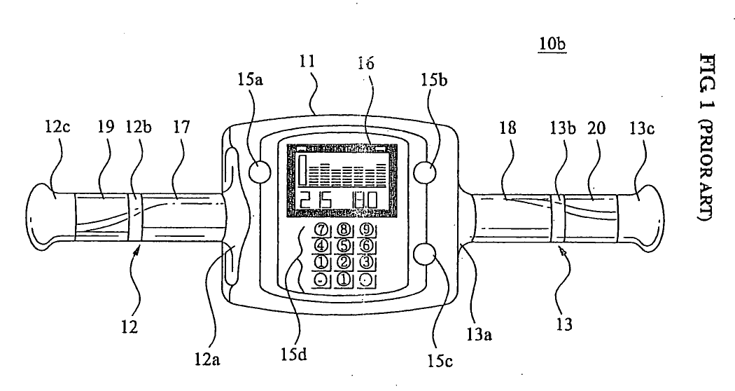

FIG. 2 is a perspective view illustrating a state of using the prior art device shown in FIG. 1;

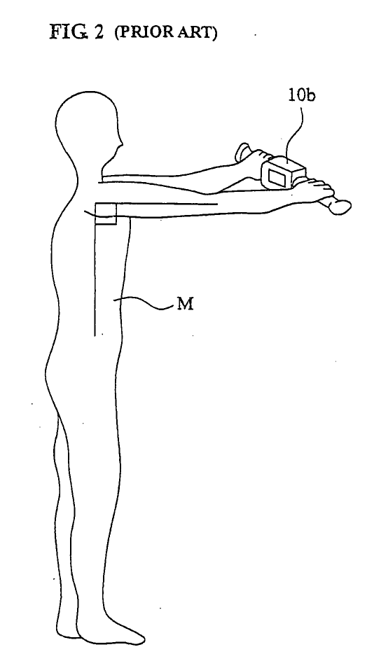

FIG. 3 is a perspective view illustrating a bio-signal measurement device according to an embodiment of the present invention;

FIG. 4 is a perspective view illustrating another example of the bio-signal measurement device according to an embodiment of the present invention;

FIG. 5 is a perspective view illustrating a state of using the bio-signal measurement device according to an embodiment of the present invention;

FIG. 6 is a configuration diagram of a health management system according to an embodiment of the present invention;

FIG. 7 is a flowchart of a health management method according to an embodiment of the present invention;

FIG. 8 is a flowchart illustrating an example of an inquiry procedure according to whether a calorie tracker exists, in the health management method according to an embodiment of the present invention;

FIG. 9 is a flowchart illustrating an example of an inquiry procedure related to blood pressure/blood sugar, in the health management method according to an embodiment of the present invention;

FIG. 10 is a flowchart illustrating an example of an inquiry procedure according to body weight/body fat, in the health management method according to an embodiment of the present invention;

FIG. 11 is a diagram illustrating an example of a daily health report, in the health management method according to an embodiment of the present invention;

FIG. 12 is a diagram illustrating an example of a weekly health report according to the analysis of weekly measured values, in the health management method according to an embodiment of the present invention;

FIG. 13 is a diagram illustrating an example of a weekly health report according to the analysis of weekly lifestyle, in the health management method according to an embodiment of the present invention;

FIG. 14 is a diagram illustrating an example of a monthly health report according to the analysis of monthly measured values, in the health management method according to an embodiment of the present invention;

FIG. 15 is a diagram illustrating an example of a monthly health report according to the analysis of monthly lifestyle, in the health management method according to an embodiment of the present invention; and

FIG. 16 is a diagram illustrating an example of a three-month trend analysis report, in the health management method according to an embodiment of the present invention.

### DETAILED DESCRIPTION OF THE PREFERRED EMBODIMENTS

Reference will now be made in detail to the embodiments of the present invention, examples of which are illustrated in the accompanying drawings, wherein like reference numerals refer to the like elements throughout. The embodiments are described below to explain the present invention by referring to the figures.

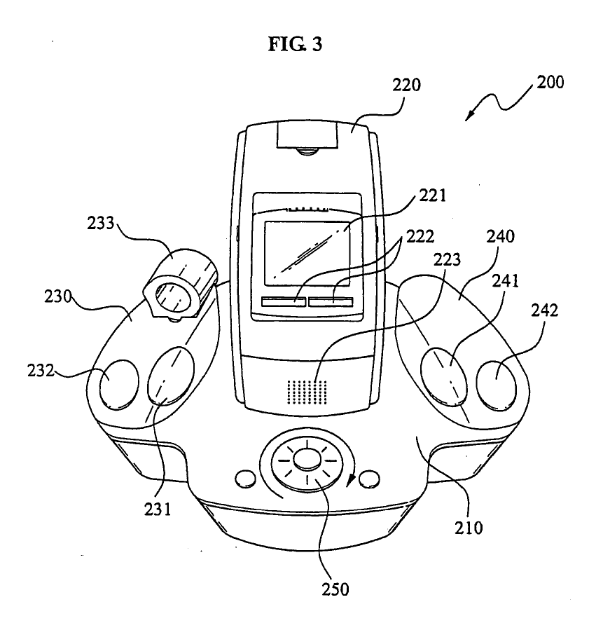

FIG. 3 is a perspective view illustrating a bio-signal measurement device 200 according to an embodiment of the present invention. As illustrated, a main body of the bio-signal measurement device 200 includes a horizontal case 210 and a vertical case 220 vertically protruded from the middle of the horizontal case 210.

Palm support portions 230 and 240 on which both hands are to be placed protrude from the horizontal case. The width of each of the palm support portions 230 and 240 is formed to be smaller than the width of a palm of an average adult so that the thumb is naturally placed on the horizontal case 210 instead of on the palm support portions 230 and 240 when each of the electrodes 231, 232, 241, and 242 is in contact with the palm of the user.

The palm support portions 230 and 240 include a left palm support portion 230 on which the left palm is placed and a right palm support portion 240 on which the right palm is placed, which are separated from each other by the shoulder width of an average adult.

A blood pressure cuff 233, in which a part of a finger is inserted to measure blood pressure, is installed on the left palm support portion 230. First and second electrodes 231 and 232 are formed below the blood pressure cuff 233. The left palm support portion 230 may be angled at a side surface of the support portion 230. The first electrode 231 may be formed to be angled according to the side surface of the angled left palm support portion 230 in order to be able to come into contact with the thenar of the user. Since the thenar is the most protruding portion of a palm of a human, reliable contact with the first electrode 231 may be maintained using the thenar. The outer surface of the second electrode 232 may be formed to have a curved surface corresponding to a curved surface of the palm. Larger sizes of the first and second electrodes 231 and 231 make for more stable and reliable contact between the electrodes and the palm. However, since there is a limitation in the size of the left palm support portion 230, the first and second electrodes 231 and 232 are sized so that the thenar is in closest contact as possible with the hypothenar.

The blood pressure cuff 233 includes a cuff housing forming the external of the blood pressure cuff 233 and a pressing part disposed in the cuff housing. The pressing part (not shown) is installed in the cuff housing and disposed along the inner circumference of the cuff housing. Since the cuff housing has an approximately cylindrical inner wall, the pressing part installed in the cuff housing may provide a cylindrical path in the blood pressure cuff.

When the blood pressure cuff 233 operates, the pressing part may expand to come firmly into contact with the inserted finger and may pressurize the finger by increasing air pressure more so that the pressing part of the cuff comes more firmly into contact with the inserted finger. After the pressure is increased more, the pressure in the pressing part is reduced. In a procedure of increasing and reducing the pressure, a blood pressure sensing unit installed in the pressing part may sense a change of an artery via a photoelectric sensor or a sound-wave sensor. A circuit element built into the bio-signal measurement device may compute the blood pressure of the user based on sensed data. The blood pressure cuff 233 for the finger, according to the present invention, may use a photoelectric sensor as a sensor, measure a pulse wave change corresponding to a change of the pressure via the photoelectric sensor installed in the pressing part, and compute the blood pressure of the user from a change of the pulse wave.

The blood pressure cuff 233 may be separated from the first and second electrodes 231 and 232 by a certain distance and may protrude upward such that a finger is in contact with the cuff the finger is extended. The first and second electrodes 231 and 232 may be composed of conductive material to apply voltage and current signal to the circuit element built into the bio-signal measurement device 200 in order to compute another bio-signal in addition to the blood pressure measured by the blood pressure cuff 233.

The left palm support portion 230 and the right palm support portion 240 are angled as if to converge away from a user such that both hands of the user are not bent up. Particularly, the left palm support portion 230 and the right palm support portion 240 are disposed such that the rear of the left palm support portion 230 and the rear of the right palm support portion 240 are both angled toward the center in which the vertical case 220 is located. Accordingly, when the user puts a palm on each of the left palm support portion 230 and the right palm support portion 240, a joint of a wrist is either not bent or is bent slightly inward in order to maintain a comfortable position, thereby reducing inaccurate measurement of a bio-signal caused by a change of position.

Third and fourth electrodes 241 and 242 are formed on the outside of the right palm support portion 240, equivalent with the left palm support portion 230. The third electrode 241 may be angled with respect to a folded end of a side surface of the angled right palm support portion 240 in order to be able to come into contact with the thenar of the user. Since the thenar is the most protruding portion of the palm of a human, the third electrode 241 is formed to be able to come into contact with the thenar, and the surface of the fourth electrode 242 is curved corresponding to a curved surface of the palm, which is similar to that of the left palm support portion 230.

The right palm support portion 240 is formed to have a width smaller than the width of a palm of an average adult such that the thumb is naturally placed on the horizontal case 210 and not on the right palm support portion 240 while each of the electrodes 241 and 242 are in contact with the palm of the user. In this case, another example of the right palm support portion 240 will be described as follows.

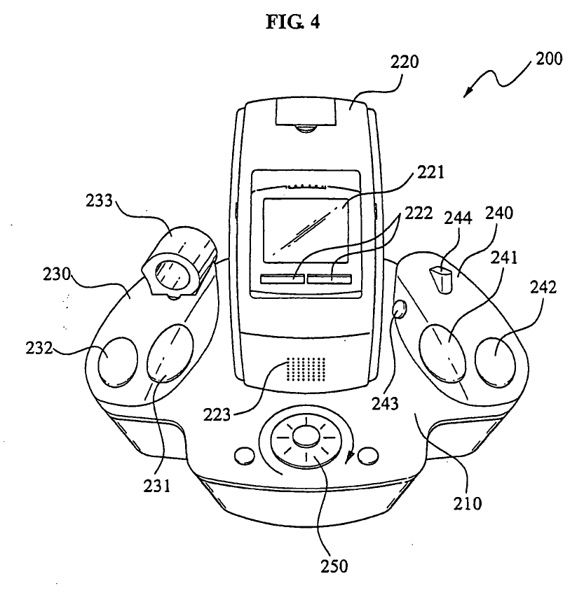

FIG. 4 is a perspective view illustrating another example of the bio-signal measurement device according to the present invention. As illustrated, a start button 243 starting the measurement of a bio-signal by a push of the user is further included. The start button 243 is located on the side of the right palm support portion 240 where the thumb is located when placed on the measurement device 200. The start button 243 is formed as a button but is not defined by that form and may be a touch-button type button.

Also, a finger guide 244 may be included, which provides a contact between two fingers and blocks the advance of the fingers. The shape of the finger guide 244 is formed as a triangular prism, but is not defined by this form and may be formed in the shape of a cylinder or other solid figure.

With respect to the left palm support portion 240, a position of the hand is approximately determined by the blood pressure cuff 233 to be able to come into contact with the electrodes 231 and 232. If the right palm support portion 240 does not include the blood pressure cuff 233, the finger guide 244 is more essential. However, the blood pressure cuff 233 does not have to be specific to the left palm support portion 230, and the finger guide 244 does not have to be specific to the right palm support portion 240. Either one may be formed on either the left palm support portion 230 or the right palm support portion 240.

A jog/shuttle wheel 250 to which information is input by the user is installed at the front surface of the horizontal case 210, between the left palm support portion 230 and the right palm support portion 240. The jog/shuttle 250 is provided as a unit for inputting information of the user according to an inquiry method that will be described later.

A display 221 providing image information to the user may be provided on the front of the vertical case 220. Input buttons 222 may be used by the user to input information in response to the information provided on the display 221 are included below the display 221. Also, a speaker 223 providing audible information to the user may be provided below the input button 222.

The blood pressure cuff 233 and the electrodes 231, 232, 241, and 242 form a sensing part to measure and transmit bio-signals to an operation unit built inside the horizontal case 210, thereby detecting the bio-signal. In this case, the bio-signals include body fat, electrocardiogram (ECG), blood pressure, heart rate, and galvanic skin response (GSR).

Hereinafter, a procedure of using the bio-signal measurement device of the present invention will be described.

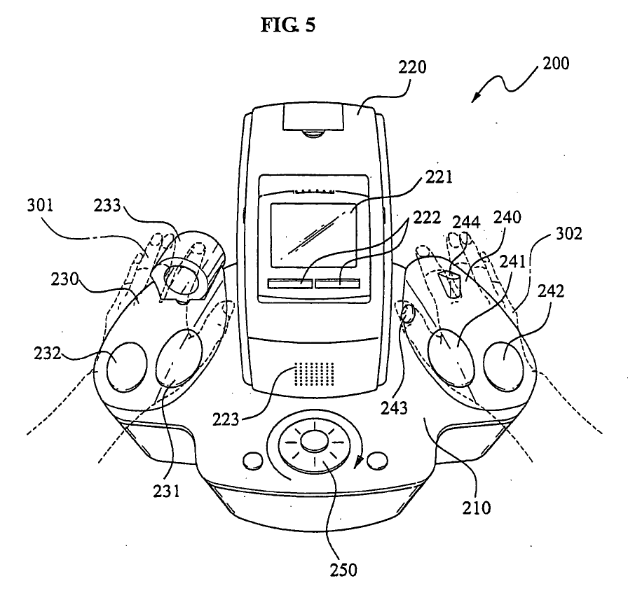

FIG. 5 is a perspective view illustrating a state of using the bio-signal measurement device according to the present invention. As illustrated, when maintaining both hands open, the user puts a left hand 301 on the left palm support portion 230 and a right hand 302 on the right palm support portion 240. Since the left palm support portion 230 is separated from the right palm support portion 240 by approximately a shoulder width, the palms may be naturally placed on the electrodes 231, 232, 241, and 242. Since a width of each of the left palm support portion 230 and the right palm support portion 240 may be smaller than a width of the hand of the user, the thumbs may be disposed on the horizontal case 210 and not on the left palm support portion 230 and the right palm support portion 240.

In this case, it is best if the user maintains open hands and the thenar of each hand 301 and 302 is in contact with the first and third electrodes 231 and 241, respectively, and the hypothenar is in contact with the second and fourth electrodes 232 and 242, respectively. The thumb is disposed on the vertical case 210, and the first electrode 231 and the third electrode 241 are formed on the front and side of the angle of each of the left palm support portion 230 and the right palm support portion 240, thereby maintaining contact with the thenar of the user.

One of the fingers of the left hand 301 is inserted into the blood pressure cuff 233 to be ready to measure the blood pressure, and the finger guide 244 is inserted between the first finger and the second finger of the right hand in order to bring the right hand 302 firmly into contact with the third and fourth electrodes 241 and 242. Also, the thumb is disposed on the horizontal case 210 to be naturally located close to the start button 243.

When the user pushes the start button 243, voltage and current are measured by each of the electrodes 231, 232, 241, and 242 and transmitted to the operation unit formed of a circuit element built into the horizontal case 210. The operation unit computes and measures body fat, ECG, blood pressure, heart rate, and GSR, and indicates such information to the user through the display 221 and the speaker 223.

As described above, not only an accurate bio-signal may be measured by providing a contact position for the palm and the electrode for both palms, but by including the blood pressure cuff, blood pressure, as well as various other bio-signals, may be quickly measured. A position of the hand may be fixed by using the finger guide, thereby reducing an error in measurement position. Further, the electrodes are located at the thenar and the hypothenar, thereby more accurately measuring a bio-signal.

Hereinafter, an inquiry method by using the present measurement device will be described as follows.

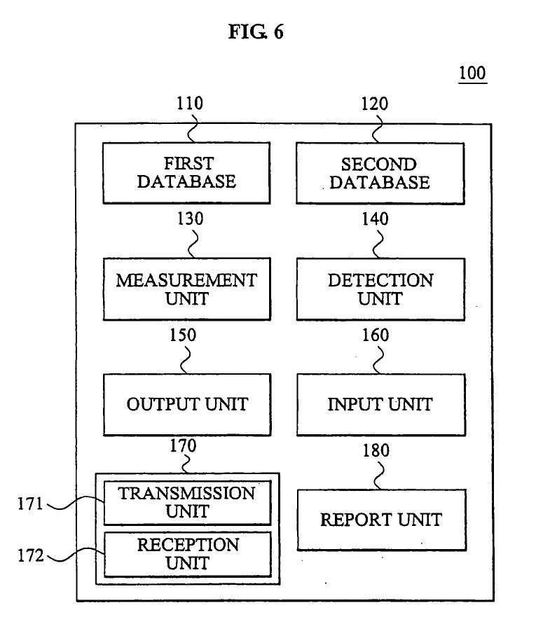

FIG. 6 is a configuration diagram of a health management system 100 according to an embodiment of the present invention.

Referring to FIG. 6, the health management system 100 includes a first database 110, a second database 120, a measurement unit 130, a detection unit 140, an output unit 150, an input unit 160, an analysis unit 170, and a report unit 180.

The first database 110 includes and maintains inquiry information. The inquiry information may include an inquiry list required in recognizing a health condition of a user. The inquiry list may include personal details inquiry information, basic inquiry information, estimation inquiry information, lifestyle inquiry information, pattern inquiry information, or health management inquiry information.

The personal detail inquiry information includes personal details and body information of the user and is provided from the user when the user registers user information in the health management system 100.

The basic inquiry information may be commonly used for testing the health condition of the user and may include a case history of the user himself, such as family history, drinking information, smoking information, and an estimation of the amount of personal activity.

The estimation inquiry information is used in all types of estimation.

The pattern inquiry information is a pattern of a health condition measurement value of the user and may be performed every three months. The pattern inquiry information can be changed to regularly scheduled periods indicating the pattern of the user's health.

The lifestyle inquiry information is in a checklist according to a user's physical condition and may be provided by measuring the user's lifestyle according to an analysis of a health condition measurement value, personal detail inquiry, and basic inquiry information. The lifestyle inquiry information may be outputted in association with the detected change in lifestyle. These checklist items must be checked when the user is at least one of: overweight, obese, morbidly obese, under hypertension care, a hypertension patient, pre-diabetic, and diabetic.

In this example, the health management inquiry information is associated with a management program for the user suffering from hypertension caused by obesity and pre-diabetes. The health management inquiry information is for measuring and inputting required information when the user takes part in a health improvement program. The health management inquiry may be used with a health consultant system in which the health management system 100 provides the health management program via a network.

The first database 110 may include health management feedback information. The health management feedback information may include a health condition of the user based on a reply to the inquiry list and a health management method based on the health condition of the user. The health management feedback information may include at least one of diet, exercise type, and activity type information for managing the health condition of the user.

The second database 120 includes the normal physical condition information of the user. The normal physical condition information of the user is used as a standard for recognizing whether the user is in a normal physical condition or abnormal physical condition and may be averaged physical condition measurement values of physical condition measurement values of the user while the user is in a normal, healthy condition.

The measurement unit 130 measures the present physical condition of the user. The measurement unit 130 may quickly measure several bio-signals by using both hands of the user. The bio-signals may include blood pressure, ECG, pulse, breath rate, and GSR. The measurement unit 130 may measure body fat and ECG of the user by using a sensor. The measurement unit 130 may measure weight, blood sugar, body temperature, and skin condition.

The detection unit 140 compares the measured present physical condition with the normal physical condition information of the user by referring to the second database 120 and detects a changed item associated with the physical condition of the user. For example, the detection unit 140 may detect that the changed item of the physical condition of the user is blood pressure where the measured present physical condition information of the user is blood pressure, and the blood pressure is higher or lower than average when compared with the blood pressure information of the normal physical condition information of the user.

The output unit 150 outputs lifestyle inquiry information associated with the detected changed item of the inquiry information by referring to the first database 110. The output unit 150 may include a display and may output the lifestyle inquiry information via the display means. The output unit 150 may include a speaker and may output the lifestyle inquiry information as a voice via the speaker.

The input unit 160 receives inquiry reply information to the outputted lifestyle inquiry information from the user. The inquiry reply information is reply information inputted by the user and based on a present condition corresponding to the lifestyle inquiry information outputted to the user.

The analysis unit 170 analyzes the health condition of the user based on the measured present physical condition and the inputted inquiry reply information.

The analysis unit 170 may include a transmission unit 171 and a reception unit 172 while connected to a predetermined health consultant system via a network. The health consultant system may be a server managed by a professional organization that manages the health of the user and consults the user. In this case, the transmission unit 171 accesses the predetermined health consultant system via a network and transmits the measured present physical condition and the inputted inquiry reply information. The reception unit 172 receives the analysis information with respect to the health condition of the user. The analysis unit 170 may more accurately analyze the health condition of the user via the received analysis information.

The report unit 180 reports the health condition of the user according to a result of the analysis of the health condition of the user. As an example, the report unit 180 may report the health condition information and the health management feedback information daily, weekly, monthly, or quarterly. The health management feedback information may include a health management plan corresponding to the present health condition, or a detailed plan of a health management method.

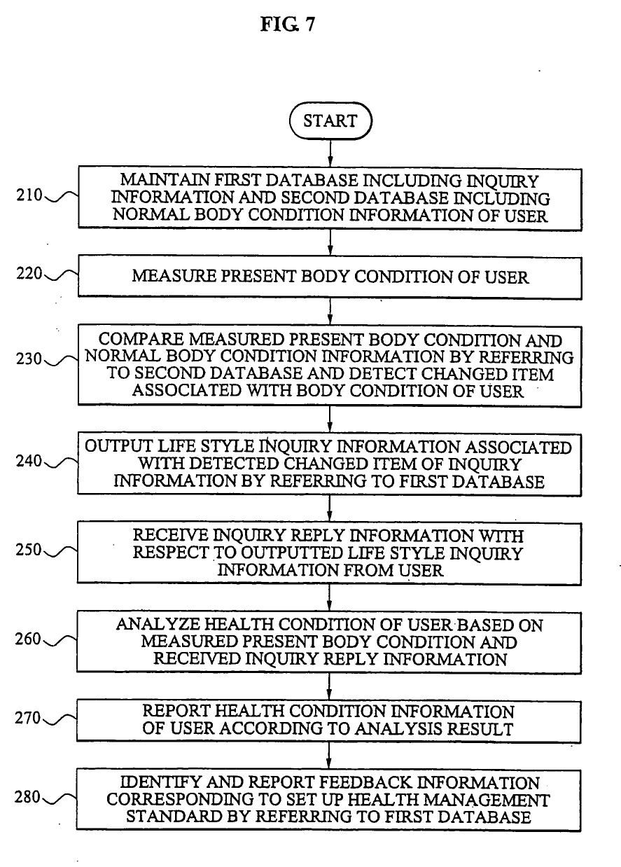

FIG. 7 is a flowchart illustrating a health management method according to an embodiment of the present invention.

Referring to FIG. 7, at 210, a health management system maintains a first database including inquiry information and a second database including the normal physical condition information of a user. The first database may record and maintain an inquiry list required in checking a health condition of the user, as the inquiry information. The inquiry list may include a personal detail information inquiry, a basic inquiry, an estimation inquiry, a lifestyle inquiry, a pattern inquiry, or a health management inquiry. The first database may further include health management feedback information. The normal physical condition information of the user may be averaged information of measurement values of the physical condition of the user. Also, the normal physical condition information of the user may be standard information for determining whether a disorder exists in the health condition of the user.

At 220, the health management system measures the present physical condition of the user. The present physical condition may include the measurement of at least one of blood pressure, blood sugar, weight, body temperature, pulse, body fat, skin condition, and amount of activity of the user.

At 230, the health management system compares the measured present physical condition with the normal physical condition information by referring to the second database and detects a changed item associated with the physical condition of the user. In the case that the measured physical condition information of the user is blood sugar information, the health management system may compare normal physical condition blood sugar information of the user with the measured physical condition information and may detect a blood sugar item as the changed item associated with the physical condition of the user if a difference exists that is more than a predefined allowance between the normal physical condition blood sugar information and the measured physical condition information.

At 240, the health management system outputs the lifestyle inquiry information associated with the detected changed item by referring to the first database. If the detected changed item is the blood sugar information, lifestyle inquiry information associated with the blood sugar is outputted. As an example, the lifestyle inquiry information associated with the blood sugar may be an inquiry for checking whether foods including sugar are ingested or an inquiry for verifying that medicine for diabetes is taken. The lifestyle inquiry information is outputted in association with the detected changed item, and other inquiry information may be outputted for each inquiry period determined according to each type of inquiry information.

At 250, the health management system receives inquiry reply information from the user in response to the outputted lifestyle inquiry information. The user may recognize the outputted lifestyle inquiry information and may input a reply to the health management system.

In operation 260, the health management system analyzes the health condition of the user based on the measured present physical condition of the user and the inputted inquiry reply information, and may set up a health management standard goal according to the analysis. If it is determined that there is a disorder in the health condition of the user, the health management system may determine a cause of the disorder with based on the measured data.

The health management system may access a predetermined health consultant system via a network and may transmit the measured present health condition and the inputted inquiry reply information to the health consultant system. The health management system receives information of the analysis of the health condition of the user from the health consultant system and may analyze the health condition of the user based on the received analysis information.

At 270, the health management system reports health condition information of the user according to a result of the analysis of the health condition of the user. The health management system may report the health condition information corresponding to the present health condition of the user daily, weekly, monthly, or quarterly.

The health condition information may include at least one of information on items used in measuring the physical condition of the user, information on a interval for measuring the physical condition of the user, information on a present health condition of the user, information on a pattern of the user's health condition, information on a value for measuring the physical condition of the user, information on the number of times of measurement of the physical condition of the user, analysis information on the value used for measuring the physical condition of the user, and lifestyle analysis information.

The user's physical condition measurement item information may be information on blood pressure, blood sugar, body weight, body temperature, pulse, body fat, and skin condition as physical condition items measured via the health management system.

The user's physical condition measurement period information may be information on the measurement interval of the physical condition of the user, measured via the health management system, for example, daily, weekly, monthly, and quarterly.

The user's present health condition information may include information such as whether the user has taken ill.

The user's physical condition measurement information may include measurement value information of the present physical condition of the user, measurement value information of a previous physical condition of the user, or averaged information of the physical condition measurement values of the user.

The user's physical condition measurement time information may indicate the number of times the physical condition of the user has been measured with the health management system.

The user's physical condition measurement value analysis information may include a result of an analysis of whether the user's present physical condition measurement value has increased or decreased compared to the user's previous physical condition measurement value information or the user's average physical condition information.

The user's lifestyle analysis information may include information analyzing lifestyle habits associated with the health of the user, such as diet, amount of exercise, and medicine dosage.

The health condition information may include a warning message indicating a dangerous situation related to the health condition of the user, and may include a cause of the analyzed disorder of the health condition. The health condition information may be shown as a diagram or a graph according to the measured present physical condition.

At 280, the health management system reports health management feedback information corresponding to the health condition information by referring to the first database. The health management system may report the health management feedback information according to the present health condition of the user daily, weekly, monthly, or quarterly.

The health management feedback information may include at least one of diet information, exercise type information, and activity type information for managing the health condition of the user.

The health management system may identify health management feedback information corresponding to the setup health management standard from the first database and may report the health management feedback information.

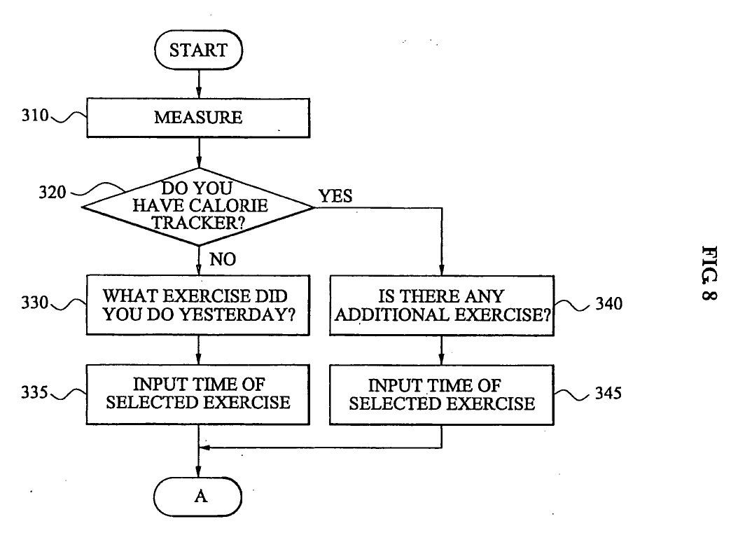

FIG. 8 is a flowchart illustrating an example of an inquiry procedure according to whether or not a calorie tracker exists, in the health management method according to the present invention.

Referring to FIG. 8, at 310, a health management system measures the present health condition of a user.

At 320, the health management system determines whether the user has a calorie tracker. The calorie tracker is an apparatus for tracking calories burned by the user via the movement of the user or heat radiation.

If the user inputs that the user does not have the calorie tracker, at 330, the health management system outputs first inquiry information for checking what exercise was performed by the user, as basic inquiry information. At 335, the health management system receives exercise time information of the exercise inputted by the user.

If the user inputs that the user has the calorie tracker, at 340, the health management system outputs second inquiry information for checking whether the user performs an additional exercise that cannot be tracked by the calorie tracker, as the basic inquiry information. At 345, the health management system receives exercise time information of the exercise selected as the additional exercise information by the user. This is required due to the limitation of calorie tracking. For example, the calorie tracker cannot be taken swimming, and is generally only valid for when the user is resting, walking and running.

The health management system may analyze calorie consumption of the user by using the exercise information and the exercise time information inputted by the user according to the first inquiry information or the second inquiry information. The health management system may measure the present physical condition and may perform an inquiry procedure regarding blood pressure/blood sugar by using a result of an analysis of the calorie consumption of the user.

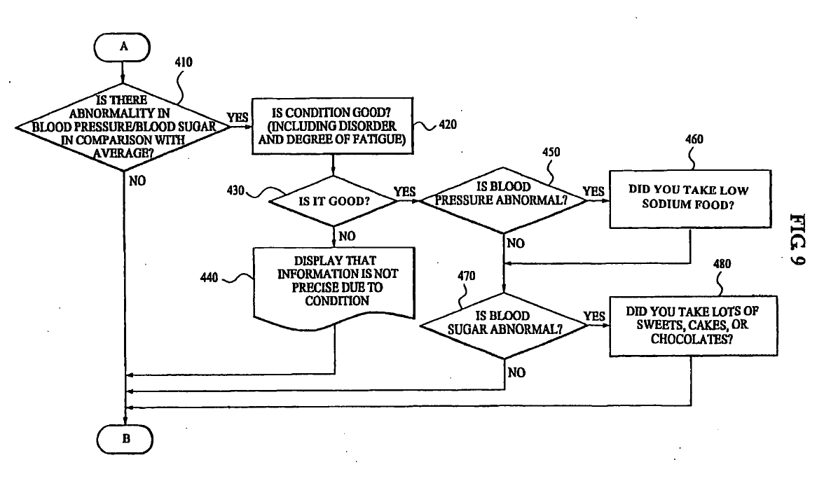

FIG. 9 is a flowchart illustrating an example of an inquiry procedure according to blood pressure/blood sugar, in the health management method according to the present invention.

At operation 410, the health management system determines whether an abnormality of the blood pressure/blood sugar exists, by comparing blood pressure/blood sugar information of the user's present physical condition information measured at 310 with blood pressure/blood sugar information of the user's average physical condition information. In this case, the health management system may determine the blood pressure/blood sugar of the user to be abnormal if the user's present blood pressure/blood sugar information is higher or lower than the user's average blood pressure/blood sugar range.

If the user's present blood pressure/blood sugar information is higher or lower than the average value range, at 420, the health management system outputs third inquiry information for checking whether the user is ill, as the basic inquiry information. The third inquiry information may request the user to check the user's present health condition by using an inquiry, for example, “Are you feeling well now?”. The third inquiry information may include inquiry information for checking a disorder and a degree of fatigue of the user. In this example, the health management system may receive information as to whether the condition of the user is good, as reply information to the third inquiry information from the user.

At operation 430, the health management system may determine whether the condition of the user is good, by using the reply information to the third inquiry information. If the condition of the user is not good, at 440, the health management system may output a message which informs the user that the information is not precise due to the current condition of the user.

If the condition of the user is good, at 450, the health management system determines whether the blood pressure of the user is abnormal when compared with the user's average blood pressure. If the blood pressure of the user is comparatively abnormal, at 460, the health management system outputs fourth inquiry information for determining whether the user ate a type of food that causes hypertension, as hypertension related inquiry information. The fourth inquiry information may include inquiry information for requesting the user to determine whether the user ate low-sodium food that does not cause hypertension, for example, “Did you eat low-sodium food?”.

At 470, the health management system determines whether the blood sugar of the user is abnormal. If the blood sugar of the user is abnormal, at 480, the health management system outputs fifth inquiry information for determining whether the user ate a type of food that may cause hyperglycemia, as diabetes related inquiry information. The fifth inquiry information may include inquiry information to determine whether the user ate food that may cause hyperglycemia, for example, “Did you eat lots of sweets?”.

The health management system may perform a procedure for a lifestyle inquiry with respect to weight and body fat in the case that the blood sugar of the user is normal.

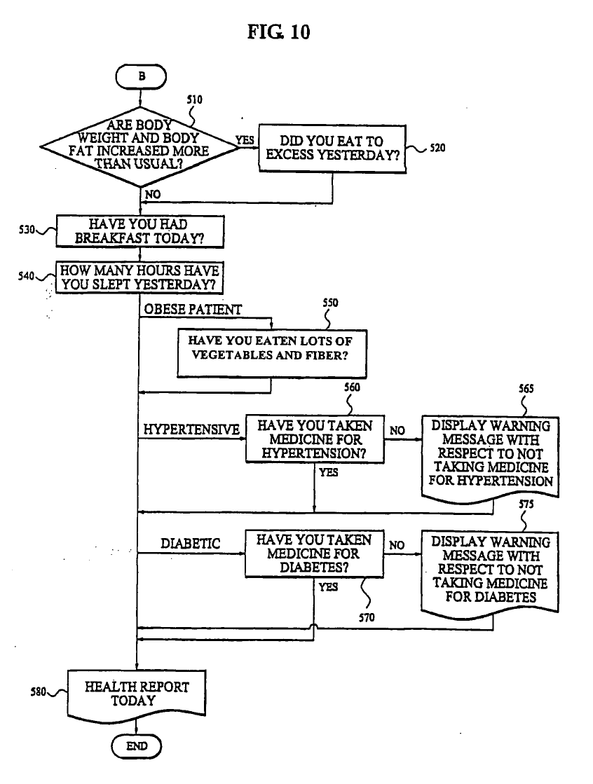

FIG. 10 is a flowchart illustrating an example of an inquiry procedure according to body weight/body fat, in the health management method according to the present invention.

Referring to FIG. 10, at 510, the health management system determines whether the body weight and body fat of the user, based on the measured user's present physical condition information, have increased more than the user's average body weight and body fat based on the average physical condition information.

If the body weight and the body fat are increased, at 520, the health management system outputs sixth inquiry information for determining a cause of the increase in the body weight and the body fat of the user, as the basic inquiry information. The sixth inquiry information may include inquiry information requesting the user to check the cause of increase of the body weight and body fat, for example, “Did you overeat yesterday?”.

At 530, the health management system outputs seventh inquiry information for determining whether the user has eaten a meal, as the basic inquiry information. The seventh inquiry information may include inquiry information requesting the user to input whether the user has eaten a meal, for example, “Have you had breakfast today?”. The health management system may receive a reply to the seventh inquiry information from the user.

At 540, the health management system outputs eighth inquiry information for determining the amount of sleep of the user, as the basic inquiry information. The eighth inquiry information may include inquiry information requesting the user to report an amount of sleep, for example, “How many hours did you sleep yesterday?”. The health management system may receive sleep time information from the user as a reply to the eighth inquiry information.

If the user is an obese patient, at 550, the health management system outputs ninth inquiry information as obesity related inquiry information. The ninth inquiry information may include inquiry information requesting the user to report whether the user has eaten foods that may help alleviate obesity, for example, “Have you eaten plenty of vegetables and fiber?”. The health management system may receive information on whether the user has eaten these foods from the user as a reply to the ninth inquiry information.

If the user is hypertensive, at 560, the health management system may output tenth inquiry information associated with hypertension to the user as hypertension related inquiry information. The tenth inquiry information may include inquiry information requesting the user to report whether the user has taken medicine for hypertension, for example, “Have you taken medicine for hypertension?”.

At operation 565, the health management system outputs a warning message with respect to not taking medicine for hypertension if information of not taking medicine for hypertension is received from the user as a reply to the tenth inquiry information. The warning message with respect to not taking medicine for hypertension may be shown to the user as a detailed method for managing hypertension, for example, “Although you are hypertensive, you are not taking medicine. In addition to controlling blood pressure, medicine is required for treating hypertension to prevent various cardiovascular diseases and other health risks. Health risks of hypertension may include kidney disease, cardiovascular disease, cerebrovascular disease, and retinal disease. Please go to the nearest health care center and consult with a medical specialist about medication and controlling hypertension as soon as possible”. Accordingly, the user may easily recognize the relevant dangers and the method of managing hypertension after checking the warning message with respect to not taking medicine for hypertension, outputted via the health management system.

If the user is diabetic, at 570, the health management system outputs eleventh inquiry information associated with glucosuria to the user, as glucosuria related inquiry information. The eleventh inquiry information may include inquiry information requesting the user to report whether the user takes medicine for diabetes, for example, “Have you taken medicine for diabetes?”. The health management system may receive information on whether the user took the medicine for diabetes from the user as a reply to the eleventh inquiry information.

At 575, the health management system outputs a warning message with respect to not taking medicine for diabetes if information of not taking medicine for diabetes is received as the reply to the eleventh inquiry information. The warning message with respect to not taking medicine for diabetes may be shown to the user as a detailed method of managing diabetes, for example, “Though you are diabetic, you are not taking medicine for diabetes. Since diabetes causes cardiovascular diseases such as ophthalmologic disease, kidney disease, and neuropathy, a test for controlling blood sugar and cardiovascular diseases is required. Meet with a medical specialist to hold a consultation and get medical attention”.

At 580, the health management system recognizes the health condition of the user according to the user's measured physical condition information and the reply information inputted by the user and outputs the daily health report corresponding to the health condition of the user.

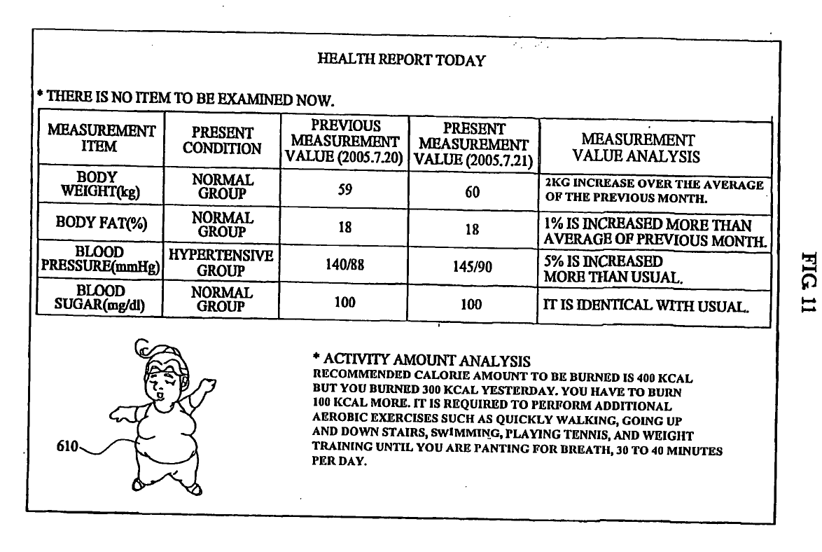

FIG. 11 is a diagram illustrating an example of a daily health report, in the health management method according to the present invention.

Referring to FIG. 11, the daily health report includes information on the physical condition of a user measured by a health management system, and information on a health condition of the user. The health condition information may be embodied as a diagram according to the measured present physical condition.

The information on the measured physical condition of the user may include body weight, body fat, blood pressure, and blood sugar of the user, as measurement items. Also, the physical condition information of the user may include the user's previous physical condition measurement value and the user's present physical condition measurement value.

The health condition information of the user may be divided into a normal group or a hypertensive group depending upon whether the present health condition of the user is normal or abnormal with respect to the corresponding measurement items. The health condition information of the user may include measurement value analysis information comparing the mean value of the user's previous physical condition measurement values with the user's present physical condition measurement value. For example, where the physical condition information measurement item is body weight, the measurement value analysis information may indicate whether the present measured body weight is more than the previously measured mean value as “A 2 kg increase in comparison with the mean value of the last month.”. The health condition information of the user may include activity amount analysis information. The amount of calories burned by the user is measured or inputted by the inquiry reply to compare the amount of the calories burned by the user with a recommended amount to be burned, thereby showing the activity amount analysis information to the user as a result of the comparison and a recommended course of action. The health condition information of the user may be embodied by an avatar 610 indicating the health condition of the user. The appearance of the avatar 610 may be changed according to the analyzed health condition of the user. The health condition analysis information of the user may be provided as to whether the user must go to a hospital or requires diagnostic information, according to the health condition of the user.

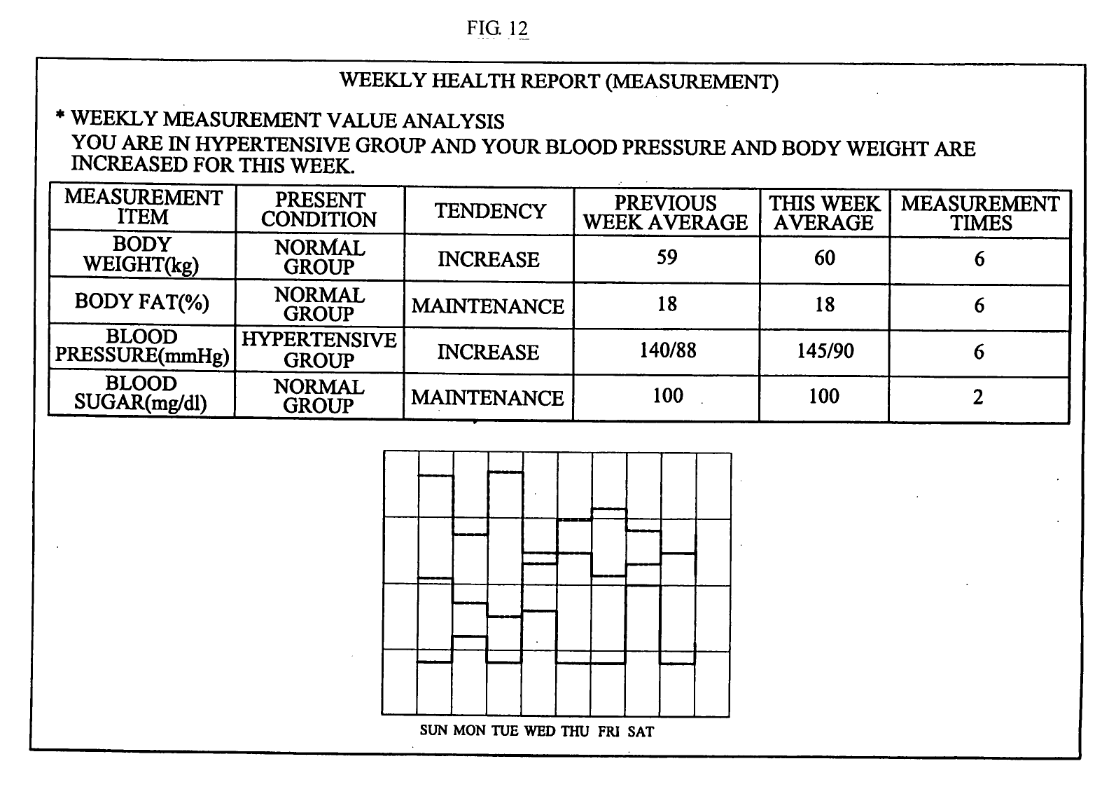

FIG. 12 is a diagram illustrating an example of a weekly health report according to a weekly measurement value analysis, in the health management method according to the present invention.

Referring to FIG. 12, the weekly health report according to the weekly measurement value analysis may show measurement items with respect to the physical condition of the user, the user's present health condition with respect to the measurement items, the pattern or trend with respect to the measurement items, the average of the measurement items of the previous week, the average of the measurement items of the current week, and the measurement number of the measurement items, as a diagram or a graph. Also, in the weekly health report, the pattern of the health condition of the user may be graphically reported as the analysis of the weekly measurement value.

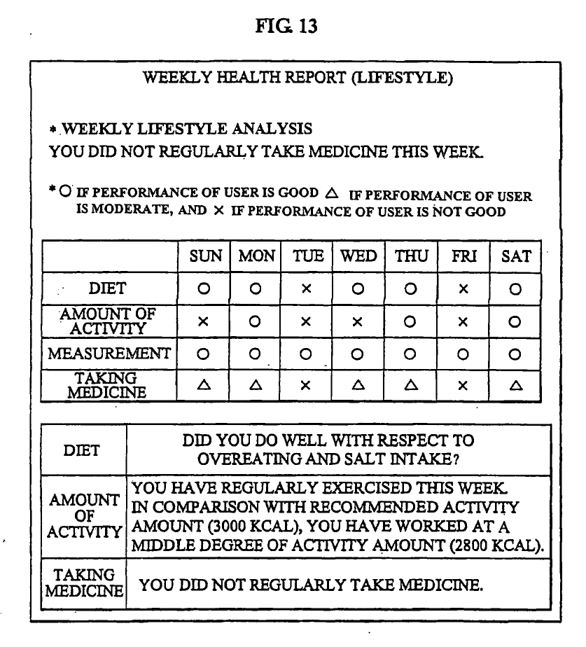

FIG. 13 is a diagram illustrating an example of a weekly health report according to a weekly lifestyle analysis, in the health management method according to the present invention.

Referring to FIG. 13, the weekly health report according to the weekly lifestyle analysis may indicate, as a diagram, diet, amount of activity, measurement or degree of completion with respect to taking medicine, as a result of analyzing the weekly lifestyle of the user. The performance of the user may be shown, for example, divided into three categories as “∘” if the performance of the user is good, “Δ” if the performance of the user is moderate, and “X” if the performance of the user is not good. Also, the result of the analysis of diet, amount of activity, and habit with respect to taking medicine may be commented on in detail. The symbols may change with respect to the choice of system.

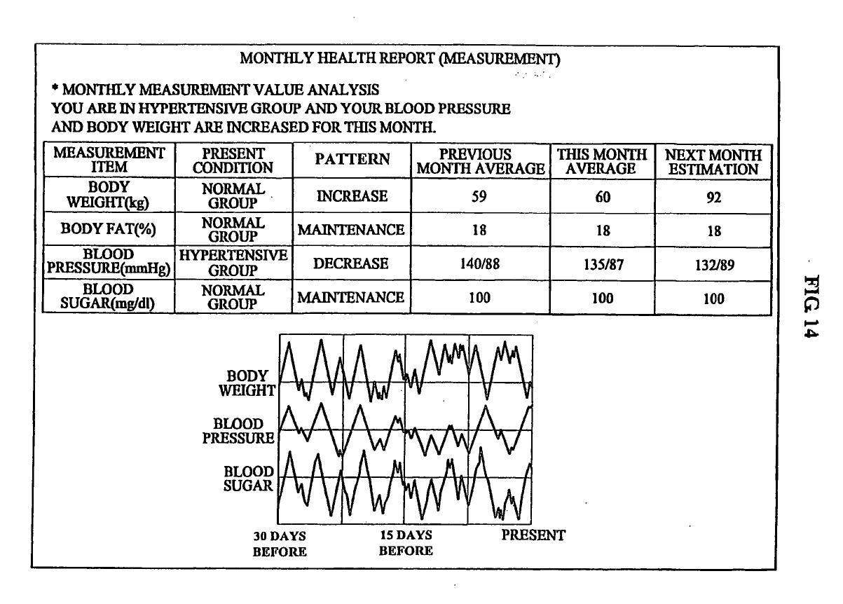

FIG. 14 is a diagram illustrating an example of a monthly health report according to monthly measurement value analysis, in the health management method according to the present invention.

Referring to FIG. 14, the monthly health report according to the monthly measurement value analysis may indicate as a diagram or a graph measurement items with respect to the physical condition of the user, user's present health condition with respect to the measurement items, pattern with respect to the measurement items, average of the measurement items of the previous month, average of the measurement items of the current month, and estimation information of the measurement items after a month. Also, in the monthly health report, the pattern of the health condition of the user may be graphically reported as the analysis with respect to the monthly measurement value.

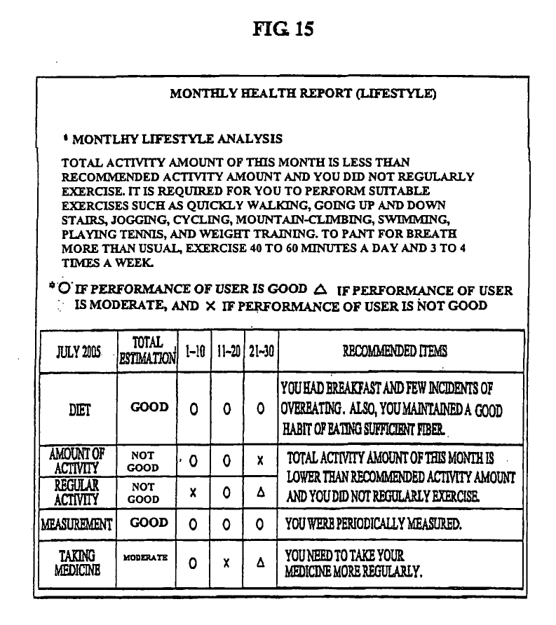

FIG. 15 is a diagram illustrating an example of a monthly health report according to a monthly lifestyle analysis, in the health management method according to the present invention.

Referring to FIG. 15, in the monthly health report according to the monthly lifestyle analysis, user's performance with respect to diet, amount of activity, regular activity, measurement or completion of taking medicine may be shown in a diagram. The user's performance is determined by the previous 10 days, and a result of the determination is divided into three categories in which “∘” indicates that the performance of the user is good, “Δ” indicates that the performance of the user of moderate, and “X” indicates that the performance of the user is not good. In addition, the user's performance may include an overall appraisal in which the performance over the previous 10 days is determined as good, moderate or not good. Also, based on the result of the analysis of the monthly lifestyle with respect to diet, amount of activity, and taking medicine, a more detailed management method according to the analysis with respect to the monthly lifestyle may be recommended.

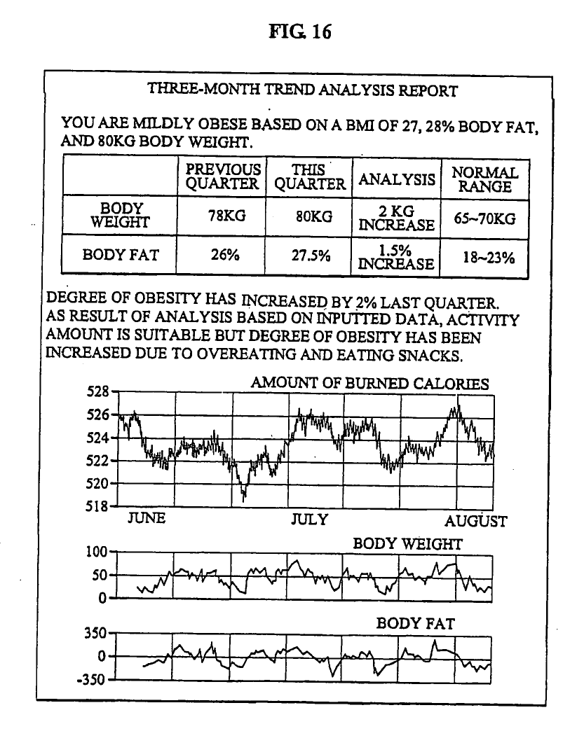

FIG. 16 is a diagram illustrating an example of a three-month trend analysis report, in the health management method according to the present invention.

Referring to FIG. 16, in the three-month trend analysis report, a result of analyzing the pattern or trend for three months, particularly with respect to body weight and body fat associated with obesity which form the measurement items associated with obesity, hypertension, and diabetes, may be shown in a diagram. In the pattern analysis report, a change for three months in the measurement items associated with obesity, hypertension, and diabetes may be shown in a graph.

Accordingly, according to the present invention, not only may a bio-signal be accurately measured by fixing a position in which both palms are in contact with electrodes while the hands are open, but also various bio-signals including blood pressure may be promptly measured. Also, according to the present invention, since a position of a hand may be fixed by using a finger guide, an error due to incorrect measurement position may be reduced.

Further, according to the present invention, since a palm may be kept in constant contact with an electrode by using electrodes located in positions accessible by the thenar and hypothenar, a bio-signal may be more accurately measured.

According to the present invention, since a health management system provides a method of minimizing a duplicated inquiry by determining physical condition information of a user, there is no duplicated inquiry information for the user to reply to.

Additionally, according to the present invention, since the user's health condition information analyzed by a health management system is provided as a diagram or graph, a user may easily visually recognize the health condition of the user.

Also, according to the present invention, since a management method according to a user's health condition analyzed by a health management system is shown in detail according to exercise, diet, and lifestyle, the user may easily manage the user's own health condition.

The embodiments of the present invention include a computer-readable medium including a program instruction for executing various operations realized by a computer. The computer-readable medium may include a program instruction, a data file, and a data structure, separately or cooperatively. The program instructions and the media may be those specially designed and constructed for the purposes of the present invention, or they may be of the kind well-known and available to those skilled in the art of computer software arts. Examples of the computer-readable media include magnetic media (e.g., hard disks, floppy disks, and magnetic tapes), optical media (e.g., CD-ROMs or DVD), magneto-optical media (e.g., floptical disks), and hardware devices (e.g., ROMs, RAMs, or flash memories, etc.) that are specially configured to store and perform program instructions. The media may also be transmission media such as optical or metallic lines, wave guides, etc., including a carrier wave transmitting signals specifying the program instructions, data structures, etc. Examples of the program instructions include both machine code, such as that produced by a compiler, and files containing high-level language codes that may be executed by the computer using an interpreter.

While this invention has been particularly shown and described with reference to preferred embodiments thereof, various changes in form and details may be made therein without departing from the spirit and scope of the invention as defined by the appended claims. Therefore, the scope of the invention is defined not by the detailed description of the invention but by the appended claims, and all differences within the scope will be construed as being included in the present invention.

What is claimed is:

1. A bio-signal detection device, comprising:
a main body having palm support portions formed on top of both sides thereof;
a sensing unit formed on at least one of the palm support portions in order to detect a bio-signal of a user and including electrodes capable of coming into electrical contact with a palm of the user; and
an operation unit within the main body and receiving a signal from the sensing unit to operate.

2. The device of claim 1, wherein the electrodes includes a first electrode capable of coming into contact with a thenar of the user and a second electrode capable of coming into contact with a hypothenar of the user.

3. The device of claim 2, wherein the first electrode is angled such that the first electrode is able to come into contact with the thenar of the user.

4. The device of claim 2, wherein the outer surface of the second electrode is designed to correspond to a curved surface of the palm.

5. The device of claim 1, wherein the sensing unit further includes a blood pressure cuff for making contact with a finger and measuring blood pressure.

6. The device of claim 5, wherein the blood pressure cuff includes:
a pressing part surrounding the finger and pressurizing the finger by using air pressure; and
a cuff housing surrounding the outer circumference of the pressing part.

7. The device of claim 5, wherein the blood pressure cuff is separated from the electrode at a certain distance and protruded upward in order to be able to come into contact with the finger while the finger is extended.

8. The device of claim 1, wherein the sensing unit further includes a finger guide designed to be interposed between fingers in order to block forward movement of the fingers when the user uses the detection device.

9. The device of claim 8, wherein the finger guide is separated from the electrode, formed in the shape of a triangular prism, and protruded from the palm support portion.

10. The device of claim 1, wherein the palm support portion protrudes from the main body.

11. The device of claim 10, wherein the palm support portion has a width smaller than a hand and has a protruded edge on which one of the electrodes is angled to be able to come into contact with the thenar.

12. The device of claim 1, further comprising a display unit providing information to the user.

13. The device of claim 12, wherein the display unit is interposed between the right and left electrodes and installed on the outside of a vertically protruding main body case.

14. The device of claim 1, further comprising a jog/shuttle wheel that the user can use to input information.

15. The device of claim 1, wherein the sensing unit further includes a start button to be pressed by the user to start detecting the bio-signal of the user.

16. The device of claim 15, wherein the start button is formed in a position in which the thumb of the user is located when the palm of the user is set on the detection device.

17. A bio-signal detection device detecting at least two bio-signals at the same time, comprising:
a horizontal case;
a vertical case vertically protruding from a center of the horizontal case;
at least two electrodes formed on opposite ends of the horizontal case, respectively, capable of coming into contact with a palm; and
a display unit installed in the vertical case.

18. The device of claim 17, wherein the electrodes includes a first electrode capable of coming into contact with a thenar of the user and a second electrode capable of coming into contact with a hypothenar of the user.

19. The device of claim 17, further comprising a blood pressure cuff installed in the horizontal case, to be able to come into contact with the finger, and used to measure blood pressure.

20. The device of claim 17, further comprising a finger guide situated between fingers of the user when the user sets the palm on the detection device and used to block forward movement of the fingers in the horizontal case.

21. The device of claim 17, further comprising at least one palm support portion formed on the horizontal case and including the electrodes formed on a top surface and a side surface of the palm support portion, wherein the palm support portion is designed to support the palm of the user.

22. The device of claim 17, wherein the horizontal case further includes a start button depressible by a user to start detection of a bio-signal, and wherein the start button is on the horizontal case in close proximity to a thumb of a user when the user places the palm on the bio-signal detection device.

23. A bio-signal detection device, comprising:
a main body;
a palm support portion formed on right and left sides of the main body;
at least two electrodes formed on a top surface of the palm support portion and capable of coming into electrical contact with a palm of a user;
a display unit installed in the main body;
a blood pressure cuff installed on the palm support portion to be capable of coming into contact with a finger to measure blood pressure; and
a finger guide formed on the palm support portion, opposite from the blood pressure cuff, and designed to be situated between two fingers to block forward movement of the fingers.

24. The device of claim 23, wherein the palm support portion protrudes from the main body.

25. The device of claim 23, wherein the palm support portion includes a first electrode capable of coming into contact with a thenar of a user and a second electrode capable of coming into contact with a hypothenar of a user.

26. The device of claim 23, wherein the blood pressure cuff is separated from the electrode and protrudes upward in order to be able to come into contact with the finger when the finger is extended.

27. The device of claim 23, wherein the display unit is interposed between the right and left electrodes and installed on an outside of a main body case vertically protruded.

28. A method of managing the health of a user in a health management system, comprising:
maintaining a first database including inquiry information and a second database including information on a normal physical condition of a user;
measuring a present physical condition of the user;
comparing the measured present physical condition with the normal physical condition information using the second database and detecting a changed item associated with the present physical condition of the user;
outputting lifestyle inquiry information associated with the detected changed item of the inquiry information using the first database;
receiving information from a reply to the outputted lifestyle inquiry information from the user;
analyzing a health condition of the user based on the measured present physical condition and the inputted inquiry reply information; and
reporting health condition information of the user according to the result of the analysis.

29. The method of claim 28, wherein the health condition information includes at least one of information on items for measuring the physical condition of the user, information on a period of measuring the physical condition of the user, information on a present health condition of the user, information on a pattern of the health condition of the user, information on a value for measuring the physical condition of the user, information on a number of measurements of the physical condition of the user, analysis information on the value for measuring the physical condition of the user, and lifestyle analysis information.

30. The method of claim 28, wherein the health condition information includes a warning message indicating a dangerous state associated with the present health condition of the user.

31. The method of claim 28, further comprising reporting health management feedback information corresponding to the health condition information by referring to the first database that further includes the health management feedback information.

32. The method of claim 31, wherein the health condition information and the health management feedback information are reported daily, weekly, monthly, or quarterly.

33. The method of claim 31, wherein the health management feedback information includes at least one of information related to diet, exercise, and a lifestyle for managing the health condition of the user.

34. The method of claim 28, wherein analyzing the health condition of the user based on the measured present physical condition and the inputted inquiry reply information further includes:
analyzing whether an abnormality associated with the present health condition of the user exists, based on the measured present physical condition and the inputted inquiry reply information; and
setting up a standard for managing health according to the result of analyzing whether an abnormality exists.

35. The method of claim 34, further comprising reporting health management feedback information corresponding to the health condition information using the first database in which the health management feedback information corresponding to the set up health management standard is stored.

36. The method of claim 28, wherein analyzing the health condition of the user based on the measured present physical condition and the inputted inquiry reply information includes:
determining whether an abnormality associated with the health condition of the user exists, based on the measured present physical condition and the inputted inquiry reply information; and
analyzing a cause of the health condition abnormality when the health condition of the user is determined to be abnormal.

37. The method of claim 36, wherein the health condition information includes the cause of the analyzed health condition abnormality.

38. The method of claim 28, wherein the present physical condition information includes at least one of blood pressure, blood sugar, body fat, pulse, breath rate, body temperature, skin condition, and the amount of activity of the user being measured.

39. The method of claim 28, wherein the health condition information is embodied by an avatar, which is changed according to the analyzed health condition of the user.

40. The method of claim 28, wherein the health condition information is shown as one of a chart and a graph, according to the measured present physical condition.

41. The method of claim 28, wherein the inquiry information includes at least one of personal details inquiry information, basic inquiry information, estimation inquiry information, lifestyle inquiry information, pattern inquiry information, and health management inquiry information, wherein the lifestyle inquiry information is outputted in association with the detected changed item and the personal details, basic, estimation, pattern and health management inquiry information are outputted for each inquiry period determined according to the type of each inquiry information.

42. The method of claim 28, wherein analyzing the health condition of the user based on the measured present physical condition and the inputted inquiry reply information includes:
accessing a predetermined health consultant system via a network and transmitting the measured present physical condition and the inputted inquiry reply information;
receiving analysis information with respect to the health condition of the user from the health consultant system; and
analyzing the health condition of the user by using the received analysis information.

43. A computer-readable recording medium in which a program for executing the method according to claim 28 is recorded.

44. A health management system comprising:
a first database including and maintaining inquiry information;
a second database including and maintaining normal physical condition information of a user;
a measurement unit measuring a present physical condition of the user;
a detection unit comparing the measured present physical condition with the normal physical condition information using the second database and detecting a changed item associated with the physical condition of the user;
an output unit outputting lifestyle inquiry information associated with the detected changed item from the inquiry information using the first database;
an input unit receiving inquiry reply information to the outputted lifestyle inquiry information from the user;
an analysis unit analyzing the health condition of the user based on the measured present physical condition and the inputted inquiry reply information; and
a report unit reporting health condition information of the user according to the result of the analysis.

45. The system according to claim 44,
wherein the first database further includes health management feedback information; and
wherein the report unit reports the health management feedback information corresponding to the health condition information by referring to the first database.

46. The system according to claim 45, wherein the report unit reports the health condition information and the health management feedback information daily, weekly, monthly, or quarterly.

47. The system according to claim 45, wherein the health management feedback information includes at least one of diet, exercise, and activity for managing the health condition of the user.

48. The system according to claim 44, wherein the analysis unit includes:
a transmission unit accessing a predetermined health consultant system via a network and transmitting the measured present physical condition and the inputted inquiry reply information; and
a reception unit receiving analysis information with respect to the health condition of the user from the health consultant system and analyzing the health condition of the user by using the received analysis information.

49. A bio-signal detection device, comprising:
a main body having sensing units on at least one side of the body and able to detect bio-signals of a user;
an input unit into which inquiry reply information is inputted; and
a display displaying information related to a health condition of the user, based on the detected bio-signals and the inquiry reply information;
wherein the inquiry reply information is inputted in response to lifestyle inquiry information output by the bio-signal detection device.

50. A method of managing a health condition of a user, comprising:
detecting bio-signals of a user sensed by at least one sensing unit of a bio-signal detection device;
receiving inquiry reply information from the user in response to outputted lifestyle inquiry information; and
outputting information related to a health condition of the user, based on the detected bio-signals and the inquiry reply information.
